(19) United States
(12) Patent Application Publication
(10) Pub. No.: US 2008/0038364 A1
(43) Pub. Date: Feb. 14, 2008

(54) Methods of processing body parts for surgery

(76) Inventors: Todd Neely, Green Oaks, IL (US); Mary Beth R. Neely, Green Oaks, IL (US)

Correspondence Address: MARSHALL A. BURMEISTER, P.O. Box 765, WILLIAMS BAY, WI, 53191, US

(21) Appl. No.: 11/654,916

(22) Filed: Jan. 18, 2007

Related U.S. Application Data
(60) Provisional application No. 60/759,898, filed on Jan. 18, 2006.

(51) Int. Cl. A61K 33/00; A01P 1/00; A61F 2/28

(52) U.S. Cl. 424/600; 623/23.63

(57) ABSTRACT

Process for a transplantable body part including extracting soluble materials therefrom, contacting the part with ozone, submerging the part in an inert body of liquid, contacting the part with nanoparticles coated with a growth factor or medicinal, and thereafter applying a pressure between 68,000 and 100,000 pounds per square inch to said body of liquid and part for a sufficient time to destroy microorganisms and affix the nanoparticles to the part. Also sterilized body parts with nonoparticles attached thereto, and body parts made by the foregoing processes.

This application is a continuation-in-part of U.S. Provisional Application Ser. No. 60/759,898 filed Jan. 18, 2006. The present invention relates to methods of processing body parts such as bone, tendons, ligaments, skin, heart valves, intestines, portions of eyes and other tissue that have been recovered from a human or animal cadaver to prepare such body parts for use in surgery as an implant, structural facilitator, or growth accelerator; and to the products produced with such methods. Further, the methods of the present invention relate particularly to processes for sterilizing bone, and to osseous products produced by such sterilization processes and nanotechnology.

### BACKGROUND OF THE INVENTION

Very significant advances in surgery have been achieved in recent years including expanded use of implants. Accordingly, a demand exists for a wide variety of transplantable body parts including bone, tendons, ligaments, skin, heart valves, intestines and parts of the eye. The invention contemplates use of the apparatus and methods of sterilization described hereinafter on all such body parts.

At the present time, processed bone for use in surgery is in great demand, and the present invention will be described in detail for such use, but it should be understood that the processes and apparatus may be used on work pieces of other body parts than bone including those described above. Considering bone alone, the worldwide orthopedic market has current sales in excess of $10 billion annually with bone replacements accounting for approximately 25% ($2.5 billion). Bone replacements include grafts and malleable putty, and such bone replacements may contain processed bone or be entirely of synthetic materials. A graft or putty is generally used by a surgeon to fill-in a space between two bone structures of a human being or animal that has resulted from trauma, pathological disease, surgical intervention or other situation where defects need to be managed in osseous surgery.

Sometimes a patient's own bone can be used for a graft or to produce bone putty. Autologous bone has been successfully used in surgeries to fill defects and is considered to be the gold standard of bone graft materials. While autologous bone is the preferred bone graft material, autologous bone grafts have a serious drawback, namely; they require the patient to undergo an additional surgical procedure and thus complicate the needed correction of the injury or disease that necessitated the bone graft. The use of allograft bone in bone replacement surgery eliminates the need for secondary surgery.

It is generally necessary to sterilize allograft bone to protect the patient from the microorganisms carried by the donor. Further, the bone marrow and mineral content of allograft bone is generally extracted to reduce the risk of incompatibility. The extracted and demineralized bone is referred to as demineralized bone matrix (DBM), and it may be used in either chunk or powdered form. Putty for use in bone surgery contains either powdered demineralized bone matrix or powdered synthetic material such as bioceramic filler, as described in U.S. Pat. No. 5,425,769 to Snyders.

Demineralized bone matrix has a proven history as a successful bone graft or bone putty material. DBM is used to promote bone growth when bone loss results from cysts, tumors, surgery, disease, fracture and other causes. Numerous clinical studies demonstrate DBM's efficacy to regenerate bone. U.S. Pat. No. 6,030,635 of Gertzman and Sunwoo filed Feb. 28, 1998 is directed to bone putty and outlines prior efforts to provide such putty for promoting bone growth at bone defect sites.

The conventional process for producing demineralized bone matrix starts with harvesting bone from the cadaver of a previously screened donor. Thereafter, the harvested bone work piece is generally subjected to total debridement, and thereafter extracted to remove the bone marrow and lipids from the bone work piece with an ethanol soak. Thereafter, the bone work piece is subjected to a hydrogen peroxide soak and then subjected to a number of washings in sterile water. Thereafter the bone work piece is subjected to terminal sterilization with ethylene oxide or gamma irradiation. Depending upon the intended use, the work piece may be thereafter subjected to chipping and milling into powder. The DBM work piece is then freeze dried either before or after packaging.

There are concerns with the use of demineralized bone matrix. One concern is that demineralized bone matrix may serve as a vehicle to transfer pathogens or viruses from the donor to the recipient. As indicated above, the bone used to produce DBM is obtained from donated bones resulting in such a possibility. The Food and Drug Administration, the American Association of Tissue Banks, and the Clinical Laboratory Improvement Amendments set standards and regulations for tissue banking procedures. Further, the United States Pharmacopeias prescribe a Sterility Assurance Level (SAL) for demineralized bone matrix of 10⁻⁶. Producers of demineralized bone matrix disagree on the level of gamma irradiation of donor bone required to achieve this Sterility Assurance Level. Different producers select irradiation levels between 9.2 kGy (9,200,000 rad) and 25 kGy (25,000,000 rad). At 9.2 kGy most pathogens are destroyed, but viruses may survive. Accordingly, the recommended standard dose for gamma irradiation of demineralized bone matrix is 25 kGy. In practice, bone tissue banks provide an irradiation dose in the range of 9.2 to 17 kGy. The American Academy of Orthopedic Surgeons reports that only five bacterial and nine viral infections have been definitely attributed to use of infected allografts. Since over the past decade, more than 5 million musculoskeletal allografts have been performed, the risk of infection is low. Nonetheless, there remains the possibility of transferring bacteria or viruses from the donor to the recipient by use of demineralized bone matrix.

Another concern in using demineralized bone matrix is that patients differ in their reaction to allografts using this material. Variations in the growth of new bone by the recipient may be explained by individual factors such as general health, age, race, diseases, metabolic rate and blood chemistry of the recipient. However, variations in bone growth by the recipient may also be explained by individual factors relating to the donor, or by differences in the processing of different allografts.

To induce bone growth and maintain the new bone in a patient, three elements are necessary, namely: scaffolding for osteoconduction; growth factors for osteoinduction; and progenitor cells for osteogenesis. Demineralized bone matrix, as presently produced, is inferior to autograft bone, and an inconsistent product. The scaffolding for osteoconduction, growth factors for osteoinduction and progenitor cells are reduced during collection and processing of the donor bone. Further, it is known that variation in the level of osteoinduction exist at present in demineralized bone matrix, but the level of scaffolding and progenitor cells is not presently tested. The inventor believes that a significant portion of the decrease in osteoconduction, growth factors for osteoinduction and progenitor cells occurs during the final sterilization step of the production process which subjects the extracted bone mass to radiation or harsh chemicals. The inventor also believes that the deleterious effect of irradiation in the final sterilization step during the production of demineralized bone matrix materially contributes to the variation between allografts, and is likely the result of variations in radiation dosage.

### SUMMARY OF INVENTION

It is an object of the present invention to provide a process for sterilizing harvested body parts which retains more of the natural growth factors that are present in the body parts as harvested than presently known methods.

Further, it is an object of the present invention to provide a process for treating harvested bone that preserves more of the osteoconduction and osteoinduction that are present in the bone as harvested than present processing methods. More specifically, it is an object of the present invention to provide a process for preparation of demineralized bone matrix that achieves sterilization of the harvested bone without the use of radiation or strong chemicals.

It is a further object of the present invention to increase the osteoconduction, and/or osteoinduction of demineralized bone matrix.

It is an object of the present invention to provide a method of processing cadaver bone that will produce demineralized bone matrix with scaffolding, and/or growth factors exceeding those of the demineralized bone matrix presently available.

More specifically, it is an object of the present invention to add supplementary scaffolding and/or growth factors to demineralized bone matrix.

The present invention assures sterilization of cadaver body parts, and specifically bone. According to one aspect of the invention, the work piece is subjected to hydrostatic pressure of at least 68,000 pounds per square inch (psi), and maintaining this pressure for a period of time sufficient to destroy bacteria, viruses, spores and other microorganisms. According to another aspect of the invention, the work piece is subjected to an atmosphere containing at least 10 percent ozone by weight for a period of 1 to 15 minutes to destroy bacteria, viruses, spores and other microorganisms. The work piece may be contacted with ozone in any step of processing including prior to or after subjection to hydrostatic pressure of 68,000 to 100,000 pounds per square inch. In a preferred mode of practicing the invention, a bone work piece is immersed in a solution of distilled water and at least 10 percent ozone by weight for a period of time sufficient to destroy bacteria, viruses, molds and spores, and thereafter the bone work piece while immersed in the ozonated distilled water solution is subjected to a hydrostatic pressure of between 68,000 and 100,000 pounds per square inch for a period of time sufficient to destroy remaining bacteria, viruses, molds and spores.

Commercially available demineralized bone matrix provides less scaffolding to promote osteoconduction than allografts of bone because the demineralization process removes scaffolding and osteoinductive growth factors. On the other hand, demineralized bone matrix has greater osteoinduction and progenitor osteogenesis than bone allografts because of the removal of the mineral content of the bone. It is therefore desirable to supplement the scaffolding of demineralized bone matrix to improve osteoconduction. In accordance with the present invention, micro particles and/or nanoparticles are admixed with distilled water or a brine solution to form a liquid mixture and the demineralized bone work piece is immersed in this mixture. Preferably, at least 10 percent ozone by weight is added to the mixture to accelerate or complete sterilization. The mixture of bone work piece, particles, dissolved ozone and water or brine solution are thereafter subjected to a hydrostatic pressure between 68,000 and 100,000 pounds per square inch for a period of time sufficient to impregnate a portion of the particles into the bone, and preferably, the hydrostatic pressure is cyclically varied to increase the portion of particles that attach to the bone work piece. The hydrostatic pressure is maintained at a pressure between 68,000 and 100,000 pounds per square inch, preferably between 78,000 and 100,000 pounds per square inch, for a sufficient time to achieve destruction of bacteria, viruses, molds and spores.

The process of demineralizing bone greatly reduces but does not eliminate bone growth factors, and the remaining growth factors are more bioavailable as a result of removal of the mineral content of the bone work piece. Nonetheless, it is desirable to supplement the growth factors in demineralized bone matrix to provide the highest levels of growth factors to promote faster bone generation and to provide more uniform products. Optionally, according to the present invention, growth factors and progenitor cells are coated on the micro particles and/or nanoparticles prior to the high pressure hydrostatic sterilization step, and also on the work piece following the sterilization step.

### DESCRIPTION OF THE DRAWINGS

The objects of this invention and other objects will become more apparent from the following description including the drawings, in which:

FIG. 1 is a schematic view of a high hydrostatic pressure sterilizer suitable for processing tissue including demineralized bone matrix according to the present invention;

FIG. 2 is a schematic view of a combination ozone and high hydrostatic pressure sterilizer suitable for processing tissue including demineralized bone matrix according to the present invention; and

FIG. 3 is a schematic view of an ozone concentration enhancing system for use in the sterilizer of FIG. 2.

### DETAILED DESCRIPTION OF INVENTION

Cortical bone and cancellous bone are currently recovered commercially from human and animal cadavers in accordance with regulations of the United States Food and Drug Administration and the American Association of Tissue Banks for use in osseous surgery. Unprocessed, raw cortical and cancellous commercially obtained bone is suitable for use as a work piece for the present inventive process; however, cancellous bone is preferred because of its greater porosity.

Bone to be processed in accordance with the present invention follows the present commercial practice for producing demineralized bone matrix up to the sterilization step. As in present commercial practices, the cadaver bone is subjected to total debridement and thereafter the bone work piece is subjected to chemical extraction to remove bone marrow and lipids, generally by an ethanol soak. Residual ethanol is then removed from the work piece, generally by a hydrogen peroxide soak followed by multiple washes with sterile water.

In accordance with one embodiment of the present invention, the bone work piece is then subjected to hydrostatic pressure of between 68,000 and 100,000 pounds per square inch to sterilize the work piece. By submerging the bone work piece in liquid and removing all gasses, and thereafter subjecting the bone work piece to hydrostatic pressure, all portions of the work piece experience the same pressure with an absence of shear forces, thus avoiding crumbling of the work piece. Suitable commercial equipment is presently available for subjecting bone work pieces to hydrostatic pressure of at least 68,000 pounds per square inch, such as the 215 L Ultra High Pressure Processing systems available from Avure Technologies Incorporated of Kent, Wash. Other companies that produce such high pressure processing equipment are Engineered Pressure Systems (USA), Elmhurst Engineering (USA), Stansted Fluid Power (UK) Mitsubishi (Japan), Kobelco (Japan), Uhde Hochdrucktechnik (Germany), ACB-Alstom (France), Stork Food and Dairy Systems (Netherlands), and Resato (Belgium).

High hydrostatic pressure has been used commercially to process food, and it is known that bacteria, viruses, molds and spores can be destroyed by subjecting them to high enough hydrostatic pressure for a sufficient period of time. The inventor has found that the osteoconductive and osteoinductive properties of bone will not be materially harmed by exposing the bone to hydrostatic pressures under about 100,000 pounds per square inch, but that the bone will be adversely affected by hydrostatic pressures in excess of 120,000 pounds per square inch. At hydrostatic pressures less than 68,000 pounds per square inch, some bacteria, viruses, mold and spores will survive for substantial periods of time. Accordingly, the inventor has determined that a bone work piece must be subjected to hydrostatic pressure of between 68,000 and 100,000 pounds per square inch for a sufficient period of time to reduce the concentration of bacteria, molds, viruses and other microorganisms to acceptable levels, and that a sufficient period of time to achieve significant sterilization is from 1 minute to 15 minutes, and preferably from 5 minutes to 15 minutes. The inventor has determined that virtually all bacteria, and most viruses, molds and spores are destroyed by subjecting a bone work piece to a pressure of at least 68,000 pounds per square inch for a period of 5 minutes.

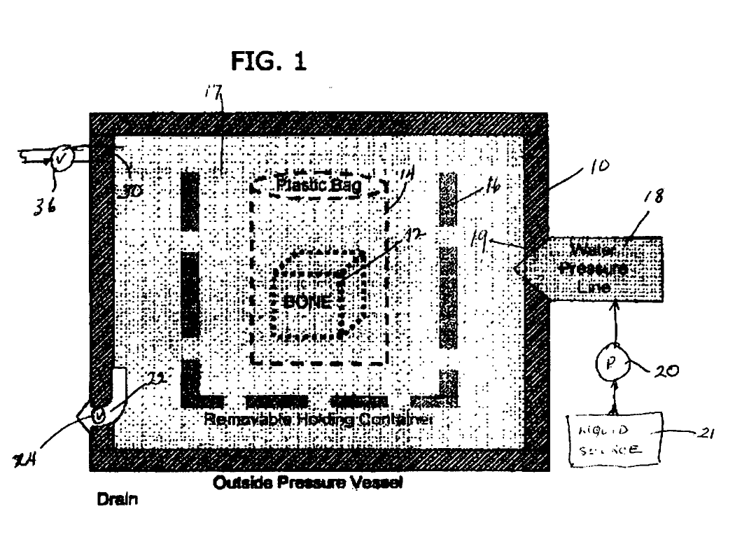

As illustrated schematically in FIG. 1, the high hydrostatic pressure processing equipment referred to above has a closed, leak proof vessel 10 which serves as a processing chamber, and the bone work piece 12 is placed within the closed vessel 10. For handling convenience, the bone work piece is enclosed within a porous plastic bag 14 and the bag is placed within a perforated, removable holding container 16 that is provided with an open top 17. The holding container 16 is used as a convenient vehicle to facilitate loading of the bone work piece outside of the vessel 10 before placing the holding container 16, bag 14 and work piece 12 within the processing vessel. The high hydrostatic pressure equipment also has a pressure line 18 which is connected to an inlet 19 communicating with the interior of the vessel 10. The pressure line 18 is also connected to a pump 20, and the pump 20 has an inlet connected to a source of liquid 21 which is preferably either sterile water or a brine solution. The pressure vessel 10 is also provided with a drain 22 near the bottom of the vessel, and the drain may be opened or closed by a valve 24. The vessel 10 is also provided with an exhaust port 30 in the upper portion of the vessel, and the exhaust port is provided with a valve 36.

With the drain valve 24 closed and the exhaust valve 36 open, the gaseous atmosphere within the vessel 10 is displaced by liquid entering the vessel through the pressure line 18. The liquid within the vessel 10 penetrates the perforated holding container 16 and the porous bag 14 to immerse the bone work piece 12. The liquid within the vessel 10 also penetrates the pores of the bone work piece 12 so that hydrostatic pressure exerted on all sides of every portion of the work piece is equal, thereby avoiding shear forces. Thereafter, the exhaust valve 36 is closed and the pump 20 activated to increase the quantity of liquid within the vessel 10, thus increasing the pressure within the pressure vessel 10 to between 68,000 and 120,000 pounds per square inch. The pressure within the pressure vessel 10 is maintained between 68,000 and 120,000 pounds per square inch for a period of 1 to 15 minutes, preferably 5 minutes at a pressure of between 68,000 and 100,000 pounds per square inch.

The temperature of the bone work piece 12 is at room temperature (about 68 degrees Fahrenheit) when it is placed in the pressure vessel 10, and its temperature rises during the period the period that pressures are established and maintained above 68,000 pounds per square inch within the vessel, but the temperature will not rise to a value high enough to damage the bone growth properties of the bone work piece. In practice, the temperature of the bone work piece 12 typically increases during processing by 20 degrees Fahrenheit, but no more than 50 degrees Fahrenheit.

Applying hydrostatic pressure between 68,000 and 100,000 pounds per square inch to the bone work piece 12 for a period of 5 minutes at room temperature destroys all bacteria, molds and spores, and most viruses on the work piece. This may be sufficient sterilization for the intended use of the work piece, and if so no further sterilization is needed. If the intended purpose of the work piece requires greater sterilization than provided by the high hydrostatic pressure process described above, or for some reason this process is not desired, the inventor employs one of the following three sterilization processes which utilize ozone, either alone or in combination with high hydrostatic pressure sterilization.

The first of the three ozone processes contacts the bone work piece with ozone, generally in a gaseous environment, for a period of time sufficient to destroy bacteria, viruses, molds and spores to the desired level, and no other process of sterilization is employed. The second of the three processes, like the first process, contacts the bone work piece with ozone, generally in a gaseous environment, for a period of time sufficient to destroy bacteria, viruses, molds and spores to a level above the desired level, and thereafter subjects the work piece to high hydrostatic pressure for a sufficient period of time to destroy bacteria, viruses, mold and spores to the desired lower level. The third process contacts the work piece with a mixture of sterile water or brine and ozone in a pressure vessel for a period of time, and thereafter raises the hydrostatic pressure within the vessel to a pressure between 68,000 and 100,000 pounds per square inch and maintains that pressure for a sufficient period of time to destroy the bacteria, viruses, molds and spores on the work piece to a sufficiently low level for the intended purpose of the work piece. The high hydrostatic pressure also destroys the ozone present in the pressure vessel.

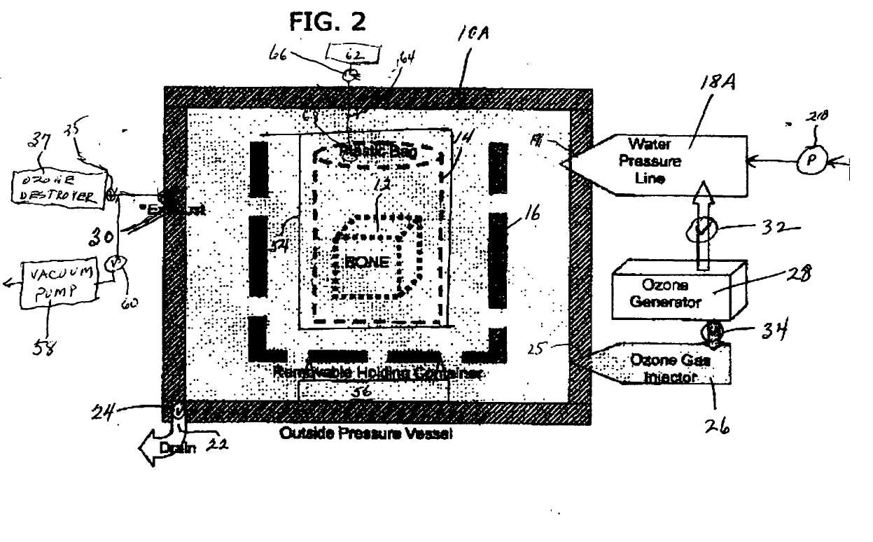

The first ozone sterilization process is described with reference to FIG. 2 which illustrates a modification of the sterilization equipment of FIG. 1. Those components of FIG. 2 which are the same as corresponding components of FIG. 1 carry the same reference numbers. The vessel 10A is a modification of the pressure vessel 10 of FIG. 1, and it is provided with an inlet 25 connected to an ozone gas injector 26 which is connected to an ozone generator 28. The vessel 10A is also provided with an exhaust port 30 which is connected to an ozone destroyer 37 through a flow control valve 35. In this process, the ozone generator 28 produces ozone gas which is conducted through the valve 34 to the ozone gas injector 26 which injects a gaseous mixture of ozone, oxygen and air into the vessel 10A to fill the vessel 10A. The drain valve 24 is closed and the exhaust valve 35 is open during the step of filling the vessel 10A with an ozone gas mixture. The gaseous atmosphere within the vessel 10A is constantly bled out of the vessel 10A through the exhaust port 30 to an ozone destroyer 37 and replaced with newly generated ozone. Ozone gas is unstable and breaks down to oxygen with a half-life of 20 minutes. For effective sterilization, the work piece must be subjected to an atmosphere of 10 to 15 percent ozone by weight, and preferably 12 to 50 percent ozone by weight, for a period of 5 to 15 minutes. Hence, ozone must be continuously generated and introduced into the vessel 10A during the period of exposure of the work piece 12.

Dielectric ozone generators are available commercially and they are suitable for use as the ozone generator 28 of the present invention. They use a high voltage source (6,000 to 20,000 volts) to create an arc between two electrically conducting plates, and provide a flow of air, or preferably oxygen, through the space between the electrically conducting plates. The ozone is generated from the oxygen by the arc. By feeding oxygen gas, rather than air, into the space between the electrically conducting plates, the generator will produce 8 to 12 percent ozone by weight with the remainder gas being largely oxygen.

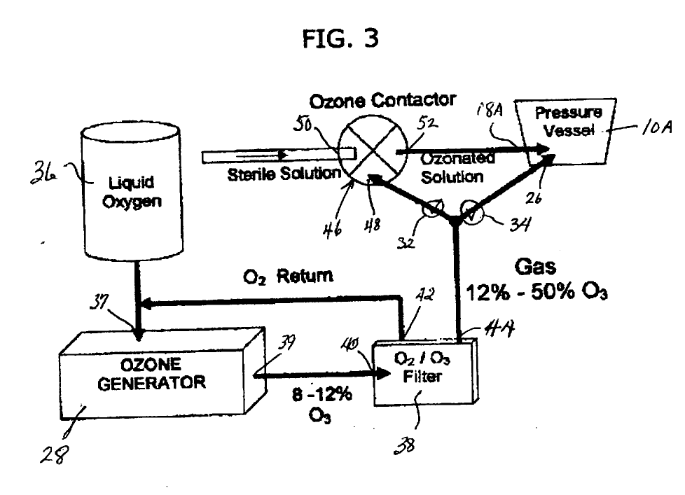

FIG. 3 illustrates a high concentration ozone generator that is capable of generating ozone gas with a minimum of 12 percent to a maximum of 100 percent ozone by weight, and may beneficially be used for the ozone gas generator 28 of FIG. 2. The high concentration ozone generator of FIG. 3 has a dielectric ozone generator 28 with a liquid oxygen source 36 connected to the intake 39 of the dielectric generator, the dielectric generator being the same ozone generator 28 illustrated in the embodiment of FIG. 2. The dielectric ozone generator has an outlet 41 connected to a filter 38 through an intake 40, and the filter 38 has two outlets 42 and 44. The filter 38 separates oxygen from ozone in the flow of gas entering the intake 40 of the filter based on the significantly higher molecular weight of ozone (48) than the molecular weight (32) of oxygen. The filter operates as a settling tank, and delivers oxygen from the upper portion of the filter to outlet 42 and ozone from the lower portion of the filter to outlet 44. Filter outlet 42 is connected to the intake 39 of the dielectric generator 28 and undergoes a further passage through the dielectric ozone generator 28. The ozone at the outlet 44 of the filter 38 is conducted to the vessel 10A of FIG. 2 through the ozone gas injector 26, or it is conducted to an ozone contactor 46 for mixing with sterile water or brine, to be discussed hereinafter. The dielectric ozone generator 28 produces about 8 to 12 percent ozone from oxygen gas entering the intake 39 of the generator, but by recirculating the exhausted oxygen from the dielectric generator through the dielectric generator, the concentration of ozone produced by the high concentration ozone generator 28 is increased. The concentration of ozone in the gas from the outlet 44 of the filter 38 is an inverse function of the flow rate. The inventor has found that ozone concentrations from 12 to 50 percent are available at the outlet 44, and such concentrations are suitable for use in sterilizing work pieces.

FIG. 3 also illustrates schematically the equipment for mixing ozone with sterile water and conveying the sterile water/ozone mixture to the pressure vessel 10A. Ozone gas from the outlet 44 of the filter 38 is conducted through the valve 32 to an intake 48 of an ozone contactor 46, the valve 34A between the outlet 44 of the filter 38 and the pressure vessel 10A being closed. The ozone contactor 46 has a second intake 50 which is connected to a source of a sterile liquid, such as sterile water or brine (not shown) and an outlet 52 connected to the pressure line 18A to the pressure vessel 10A. Ozone contactors suitable for use as contactor 46 of FIG. 3 are available commercially and diffuse small bubbles of ozone gas into a body of sterile water or brine, usually by a series of baffled chambers or a turbine diffuser, and hence require no further description.

Ozone is the second most powerful disinfectant known behind fluoride, and is 10 times more powerful than chlorine. Ozone is known to destroy bacteria, viruses and other microorganisms by contact as a gas or in a liquid mixture. However, the concentration of ozone must be greater than a threshold value and the time of exposure must be sufficient and varies for different microorganisms. The time required to destroy a particular microorganism with ozone also is dependant on the temperature. The following Table 1 shows the relationships in CT units between time, temperature and concentration of ozone required to inactivate Giardia and virus organisms. The CT unit is the ratio of the amount of ozone present in milligrams per liter times the period of exposure required to inactivate a microorganism in minutes.

TABLE I

| | Temperature (° C.) | | | | |
|---|---|---|---|---|---|
| | 5 | 10 | 15 | 20 | 25 |
| Giardia Inactivation | | | | | |
| 0.5 log | 0.32 | 0.23 | 0.16 | 0.12 | 0.08 |
| 1.0 log | 0.63 | 0.48 | 0.32 | 0.24 | 0.16 |
| 1.5 log | 0.95 | 0.72 | 0.48 | 0.36 | 0.24 |
| 2.0 log | 1.3 | 0.95 | 0.63 | 0.48 | 0.32 |
| 2.5 log | 1.6 | 1.2 | 0.79 | 0.60 | 0.40 |
| 3.0 log | 1.9 | 1.4 | 0.95 | 0.72 | 0.48 |
| Virus Inactivation | | | | | |
| 2.0 log | 0.6 | 0.5 | 0.3 | 0.25 | 0.15 |
| 3.0 log | 0.9 | 0.8 | 0.5 | 0.4 | 0.25 |
| 4.0 log | 1.2 | 1.0 | 0.6 | 0.5 | 0.3 |

* (From USEPA 1991)

Compared to other disinfectants like chlorine, chlorimine and chlorine dioxide, ozone is the strongest disinfectant and also the fastest acting.

In the second ozone sterilization process, sterilization with ozone is advantageously followed or preceded by high hydrostatic pressure sterilization, since certain microorganisms are more easily destroyed by high hydrostatic pressure than ozone and vise versa. Further, the same vessel used to subject a work piece to ozone sterilization, as described above with reference to FIG. 2, also can serve to subject the work piece to high hydrostatic pressure sterilization.

In this second embodiment of sterilization with ozone, the vessel 10A of FIG. 2 is constructed to withstand internal pressures in excess of 100,000 pounds per square inch, and as previously described a pump 20 is connected through a water pressure line 18A to the inner chamber of the pressure vessel 10A. The pump 20 is connected to a source of sterile water or saline solution 21. Also, the ozone generator 28 is connected to the water pressure line 18A through a valve 32 and to the ozone gas injector 26 through a valve 34. The exhaust port 30 also has a flow control valve 36.

To sterilize a work piece, the work piece 12 is placed in the vessel 10A, preferably in a porous plastic bag 14 disposed within a perforated removable holding container 16 and the vessel is sealed against leakage of either gas or liquid. The vessel 10A is thereafter filled with a gaseous or liquid mixture containing between 10 and 15 percent ozone by weight, oxygen and air. In a liquid mixture, the ozone, oxygen and air are mixed with sterile water or brine. The mixture within the closed vessel 10A is maintained with at least 10 percent ozone by weight for a period of 1 to 15 minutes, preferably 5 to 15 minutes, to destroy bacteria, viruses, molds and spores that may be present on the work piece. Preferably, liquid is disposed within the vessel 10A, and comprises of a mixture of ozone, oxygen, air and sterile water or brine, and the liquid may include other ingredients which promote the destruction of bacteria, viruses, molds and spores, such as a surfactant and a chelating agent.

Following the period in which the work piece is contacted with ozone, the flow of ozone is cut off by closing the valves 32 and 34 from the ozone generator and the exhaust valve 35. The drain valve 36 remains closed. The hydrostatic pressure within the vessel 10A is now raised to a pressure of between 68,000 and 100,000 pounds per square inch by adding sterile water or brine through the pump 20 and water pressure line 18A into the vessel 10A. This pressure is maintained for a period of 1 to 15 minutes, preferably 5 minutes to complete the sterilization of the work piece.

The third alternative process for using ozone in sterilizing a work piece also can use the equipment illustrated in FIG. 2. In this process, the work piece 12 is placed within a porous bag 14, positioned on a perforated removable holding container 16 and placed within the vessel 10A. The drain 22 is closed by the valve 24, and the vessel 10A is filled with a liquid mixture of ozone, oxygen, air and sterile water or brine at about 20 degrees Celsius. Air trapped within the vessel 10A during the filing process is bled from the vessel through the exhaust port 30. The liquid mixture entering the vessel 10A contains between 12 and 50 percent ozone by weight. When the liquid mixture comprising sterile water or brine and ozone fills the vessel 10A, the exhaust valve 36 is closed and the drain valve 24 is partially opened to permit a slow flow of liquid from the vessel 10A. The pump 30 supplies sufficient ozonated liquid to the vessel 10A to replace the liquid passing through the partially closed drain valve 24 to maintain the ozone level within the vessel 10A substantially constant for a sufficient period of time to destroy bacteria, viruses, molds and spores, for example a period of 5 to 15 minutes. Thereafter, the flow of ozonated liquid into the vessel 10A is terminated by closing the valve 32 to the ozone generator 28, shutting down the pump 20 and closing the drain valve 24. Thereafter the pump 20 is reactivated to deliver sterile water or brine into vessel 10A to increase the hydrostatic pressure within the vessel to between 68,000 and 100,000 pounds per square inch at a temperature between 10 and 70 degrees Celsius. This pressure is maintained for a period of 5 to 15 minutes to expose the work piece to high hydrostatic pressure, thus further destroying bacteria, viruses, molds and spores on the work piece. Also the application of pressure within vessel 10A will destroy the ozone remaining in the vessel 10A.

Both high hydrostatic pressure and ozone are effective in destroying bacteria and viruses, but as presently understood not in the same way. Ozone penetrates the outer shell of bacteria and oxidizes the outer protein coat of viruses to effect destruction, while high hydrostatic pressure interferes with the metabolism of bacteria and viruses. By contacting bacteria and viruses with ozone and subjecting them to high hydrostatic pressure, the ozone process destroys part of the microorganism population and weakens the rest of the microorganism population to facilitate destruction by high hydrostatic pressure. High hydrostatic pressure sterilization may precede or follow ozone sterilization with equal effectiveness.

As indicated above, the bone growth that will be achieved with a demineralized bone matrix allograft may be accelerated by increasing the scaffolding on the demineralized bone matrix. The inventor has found that nanoparticles and microparticles may be affixed to demineralized bone matrix to add scaffolding, and that demineralized bone matrix prepared in this manner provides greater osteoconduction than demineralized bone that is not provided with attached nanoparticles or microparticles. Further, the inventor has found that nanoparticles and microparticles may be affixed to a bone work piece in the process of producing demineralized bone matrix by pressing them into the pores of the bone work piece undergoing high hydrostatic pressure sterilization and/or exposure to vacuum.

Bone is a relatively hard yet lightweight composite material formed mostly of calcium phosphate in the chemical arrangement termed hydroxyapatite. Bone is classified as either cancellous (spongy) or cortical (outer layer) bone. Cancellous bone is significantly more porous than cortical bone, and for this reason is preferred for demineralized bone matrix. Pore size in bone range from 50 to 500 micrometers which is optimal for interface activity, bone in growth, and implant reabsorbtion.

The inventor employs particles with cross-sections shorter than the size of the pores of the bone work piece being processed for demineralized bone matrix. Further, the pores of each work piece vary in size over a wide range. Hence, the inventor employs particles of a wide range of sizes to be anchored within the pores of a bone undergoing processing to demineralized bone matrix. The smallest particles used for scaffolding are carbon nanotubes which have diameters ranging from 5 to about 15 nanometers (one-thousandth of a micrometer) and lengths over 100 nanometers. To improve the scaffolding of cancellous bone, which has larger pores than cortical bone, larger particles are employed including metals and particularly titanium or silver. Titanium has been proven to promote bone growth. The diameters of the large particles range from 10 to 300 micrometers.

It is also necessary to improve the osteoinductive properties of a bone work piece being processed for demineralized bone matrix if the rate of bone growth is to be optimized and if demineralized bone matrix allografts are to produce more uniform results. The osteoinductive properties of bones processed for demineralized bone matrix in accordance with the present invention are improved and substantially standardized by adding growth factors and bone morphogenic proteins to the bone work piece during processing to demineralized bone matrix. A coating of growth factors and morphogenic proteins is applied to the nanoparticles and microparticles before these particles are affixed within the pores of the bone work piece during production of demineralized bone matrix. Nanoparticles, microparticles, growth factors including bone morphogenic proteins are available commercially and will not be further described.

The process of producing demineralized bone matrix with affixed nanoparticles and microparticles that have been coated with growth factors and bone morphogenic proteins is preferably performed with a modified construction of the apparatus illustrated in FIG. 2. In addition to the construction of the high pressure hydrostatic sterilizer equipment of FIG. 2 described above, a vacuum pump 58 is connected to the exhaust port 30 through a valve 60. The valve 35 remains inserted between the exhaust port 30 and the ozone destroyer 37.

The process of producing demineralized bone matrix with affixed nanoparticles and microparticles includes the following steps. First, cadaver bone is obtained from a licensed tissue bank. Also, microparticles and nanoparticles of carbon, silver or titanium are obtained from commercial sources. In addition, osteoinductive growth factors, neutral coating material and sterile water are obtained from commercial sources.

Second, the cadaver bone work piece is subjected to total debridement, cleaned, extracted and prepared in the conventional manner for sterilization.

Third, in the laboratory, the bone work piece, illustrated with the reference numeral 12 in FIG. 2, is placed within the perforated bag 14, and this assembly is placed within the perforated holding container 16.

The fourth step is optional. This step subjects the work piece to an ozone atmosphere to sterilize the work piece according to the process described above. To subject the work piece to ozone sterilization, the holding container 16 with its contents is placed within the vessel 10A, and valves 20, 22, 32 and 60 are closed. Valve 34 is opened to conduct ozone from the ozone generator 28 through the ozone gas injector 26 into the vessel 10A, and a small flow of ozone passes through the exhaust port 30 to the ozone destroyer 37 to prevent the atmosphere within the vessel 10A from escaping for the protection of the operator. The ozone generator 28 is activated, and the valve 34 between the ozone generator 28 and the ozone gas injector 26 is opened, thus causing ozone from the ozone generator to flow into and fill the vessel 10 The ozone concentration within the vessel 10A increases to at least 10 percent by weight and is maintained at this level for a period of 5 to 15 minutes. At the end of the period that the concentration of ozone gas is maintained in the vessel, the ozone generator 28 is inactivated; the ozone gas within the vessel is flushed with air through the exhaust port 30 to the ozone destroyer 37.

The fifth step is also optional. This step subjects the work piece to a vacuum in order to impregnate the work piece with nanoparticles and microparticles. To vacuum impregnate the work piece using the apparatus of FIG. 2, the holding container 16 with bone work piece 12 disposed within the perforated water permeable bag 14 and the bag 14 disposed within the water impermeable bag 54 is placed within the vessel 10A, and valves 20, 22, 32 and 35 are closed. The topps of bags 14 and 54 are open. Also, a tank 62 is mounted on the exterior of the vessel 10A, and provided with a port with a valve 64, and a tube 66 extends from the valve 64 into the vessel 10A and has an open end 68 disposed above the open permeable bag 14.

The vacuum pump 58 is activated and the valve 60 opened to draw gasses from the vessel 10A and exhaust them to the atmosphere. A vacuum between 0 and 30 inches of Hg is thus created within the vessel 10A. Air in the pores of the work piece 12 is removed by the vacuum pump 58 causing the work piece to expand.

A mixture of nanoparticles, microparticles and water is poured into the tank 62, and while maintaining a vacuum within the vessel 10A, the valve 66 of the tank is opened permitting the contents to flow through the tube 64 into the permeable bag 14. Air introduced into the vessel from the tank 62 continues to be exhausted by the vacuum pump 58 during the flow of the mixture into the permeable bag 14 until the vessel is completely filled with the mixture. When the vessel 10A is substancially filled with the mixture of nonoparticles, microparticles and ionized water, the vacuum pump is deactivated, and the valve 60 is closed, and any gas disposed within the vessel 10A is removed. At this time, the pressure within the vessel has returned to approximately atmospheric pressure, and the work piece 12 has returned to its original smaller size, trapping nonopaticles and microparticles in its pores during the process. Optionally, the vessel 10A may thereafter be subjected to high hydrostatic pressure, as described hereinafter, without removing the mixture of nanoparticles and microparticles, thereby further impregnating the particles into the work piece.

The sixth step is also optional. Nanoparticles and microparticles can function as carriers to deliver growth factors, morphogenic proteins, antibiotics and other compounds to the site of a surgical procedure. To do so, it is necessary to treat the nanoparticles or microparticles before they are transported to the work piece. For a bone work piece, the microparticles of titanium or silver and nanoparticles of carbon are poured into a liquid mixture of bone growth factors and morphogenic proteins to coat the exterior surfaces of the microparticles and nanoparticles. Thereafter, the liquid is drained from the nanoparticles and microparticles, and they are contacted and mixed with a neutral coating material to protect the layer of bone growth factors and morphogenic proteins.

Seventh, the bone work piece 12 disposed within the perforated bag 14 is thereafter inserted within the water permeable bag 14 through the open top thereof, and the bag 14 and its contents is placed within the water impermeable bag 54 that is also open at the top. Thereafter, a mixture of coated microparticles of titanium and silver, coated nanoparticles of carbon, and sterile water is added to the open perforated bag 14. The open bag 54 and its contents is then placed in the removable perforated holding container 16.

Eighth, the removable perforated holding container 16 with its contents is then placed on an agitating device 56 within and at the bottom of the vessel 10A. The agitating device continuously functions to maintain the nanoparticles and microparticles in suspension within the body of sterile water in the perforated bag 14 within the sealed water impermeable bag 54.

Ninth, the drain valve 24 is closed and the pump 20 is activated to fill the vessel 10A with sterile water, and thereafter the valve 35 at the exhaust port 30 is closed to cause the hydrostatic pressure within the vessel 10A to increase to between 75,000 and 100,000 pounds per square inch. The hydrostatic pressure is maintained above 68,000 pounds per square inch for a period of 5 minutes. The drain valve 24 is cyclically operated during this 5 minute period at a rate greater than once every minute to open just sufficiently to drop the pressure within the vessel 10A by about 7,000 pounds per square inch, and thereafter the drain valve 24 is immediately closed to permit the pump 20 to add sterile water to the vessel 10A and restore its higher operating pressure. The high hydrostatic pressure forces the coated nanoparticles and microparticles into the pores of the bone work piece and anchors them in place. By cyclically varying the pressure in the vessel 10A, the mixture of sterile water, coated nanoparticles and coated microparticles is continuously agitated and a larger proportion of the nanoparticles and microparticles are lodged within pores of the bone work piece than are lodged by constant level high hydrostatic pressure.

Tenth, after the five minute exposure to high hydrostatic pressure, the holding container 16 with its contents is removed from the vessel 10A. The demineralized bone matrix can be stored and shipped in the sterile liquid mixture within the impermeable bag 54, or the water impermeable bag 54 may be opened and the perforated bag 14 with its contents is removed. If the bag 54 is opened, it is thereafter dried with its contents by the application of low heat. The demineralized bone matrix 12 may be stored in its sterile porous plastic bag 14, or removed from the bag 14 for further processing or packaging.

While the foregoing description focuses on processing bone for demineralized bone matrix, many other uses for the invention are contemplated. As stated above, the present invention may be used for processing other tissue such as tendons, ligaments, skin, heart valves and intestines. Also the present invention may be used to process foods, such as meat, vegetables, fruits, fish and crustaceans. Accordingly, the present invention is not limited by the foregoing disclosure, but rather only by the appended claims

The invention claimed is:

1. The method of processing body parts from an animal or human being for transplant into an animal or human being comprising the steps of subjecting a body part to at least one solvent to extract blood, cells and other solvable materials to produce an extracted body part work piece, thereafter submerging the work piece in an inert body of liquid, and thereafter pressurizing the body of liquid to a pressure of between 68,000 and 100,000 pounds per square inch and applying said pressure to said body of liquid for a sufficient period of time to destroy bacteria, mold, spores, viruses and other microorganisms associated with the work piece.

2. The method of processing body parts comprising the steps of claim 1 wherein the body part comprises bone, tendon, ligament, skin, heart valve, intestine, or a portion of an eye from an animal or human cadaver.

3. The method of processing body parts comprising the steps of claim 1 wherein the inert body of liquid is a member of the group distilled water and brine solution,

4. The method of producing demineralized bone matrix comprising the steps of subjecting a work piece of bone to at least one solvent to extract blood, cells and minerals from the work piece, thereafter submerging the work piece in an inert body of liquid, and thereafter pressurizing the body of liquid and submerged work piece to a pressure between 68,000 and 100,000 pounds per square inch and applying said pressure to said body of liquid and work piece for a sufficient period of time to destroy bacteria, mold, spores, viruses and other microorganisms associated with the work piece.

5. The method of producing demineralized bone matrix comprising the steps of claim 4, and after extracting the work piece contacting said work piece with at least one ingredient to disinfect the work piece of bacteria, viruses, mold, spores and other microorganisms.

6. The method of producing demineralized bone matrix comprising the steps of claim 5 wherein the at least one ingredient to disinfect the work piece of bacteria, viruses, mold, spores and other microorganisms is ozone.

5. The method of producing demineralized bone matrix comprising the steps of claim 4 wherein the at least one ingredient to disinfect the bone mass includes a surfactant.

6. The method of producing demineralized bone matrix comprising the steps of claim 4 wherein the at least one ingredient to disinfect the bone mass includes a chelating agent.

7. The method of producing demineralized bone matrix comprising the steps of claim 2 and contacting the extracted bone mass with a mass of nanoparticles.

8. The method of producing demineralized bone matrix comprising the steps of claim 7 wherein the step of contacting the extracted bone mass with a mass of nanoparticles is performed prior to the step of submerging the extracted mass in a body of liquid.

9. The method of producing demineralized bone matrix comprising the steps of claim 7 wherein the nanoparticles of the mass are of carbon.

10. The method of producing demineralized bone matrix comprising the steps of claim 7 wherein the microparticles of the mass contain titanium.

11. The method of producing demineralized bone matrix comprising the steps of claim 2 and contacting the extracted bone mass with a mass of nanoparticles.

12. The method of producing demineralized bone matrix comprising the steps of claim 7 wherein the nanoparticles are coated with bone growth factors.

13. An implant for gsurgically insertion into an animal or human being comprising a body part recovered from a human or animal cadaver from which blood, cells and other solvable materials have been chemically extracted, and sterilized in an inert body of liquid at a pressure of between 68,000 and 100,000 pounds per square inch for a sufficient period of time to destroy bacteria, mold, spores, viruses and other microorganisms associated with the work piece.

14. An implant for gsurgical insertion into an animal or human being comprising claim 13 wherein a plurality of nanoparticles are disposed within the pores of the body part.

15. An implant for gsurgical insertion into an animal or human being comprising claim 14 wherein the body part is bone.
